(19) United States
(12) Patent Application Publication
(10) Pub. No.: US 2006/0064144 A1
(43) Pub. Date: Mar. 23, 2006

(54) Programmable multifunction table lamp for light therapy

(76) Inventors: Joshua Q. Chen, Newport Beach, CA (US); Yu Wei Huang, Newport Beach, CA (US)

Correspondence Address: Joshua Q. Chen;Nature Bright Company, 17th Floor, 2600 Michelson Drive, Irvine, CA, 92612, US

(21) Appl. No.: 11/158,642

(22) Filed: Jun. 21, 2005

Related U.S. Application Data
(60) Provisional application No. 60/582,857, filed on Jun. 25, 2004.

(51) Int. Cl. A61N 5/06

(52) U.S. Cl. 607/90

(57) ABSTRACT

A lamp for providing light therapy includes an input circuit, an output circuit, and a signal processing circuit. The input circuit may include a user input for providing control signals to the signal processing circuit based on input at the user input from a user. The output circuit may include a light output configured to emit light at a level of at least 2,500 lux. The light output may include one or more white LEDs and may also include one or more color LEDs. The signal processing circuit is connected to the input circuit and the output circuit, and receives the control signals from the input circuit to control the output circuit based on the control signals. A memory may be provided in communication with the signal processing circuit. The memory may store a plurality of output modes, such as a light therapy mode. The light output may include a diffuser that is configured to diffuse the high-intensity light for effective use.

### CROSS REFERENCE TO RELATED APPLICATION

The present application claims priority on U.S. Provisional Application for Patent Ser. No. 60/582,857, the entire disclosure of which is incorporated herein by reference.

### BACKGROUND OF THE INVENTION

The present invention related to lighting devices such as table lamps. The invention also related to devices for performing light therapy and dawn-and-dust simulation.

Recent research finds that most people in the northern United States and Europe experience seasonal changes in mood and behavior, also known as seasonality. In its most marked form, affecting an estimated six percent of the population in the United States, seasonality can actually cause a great deal of distress and difficulties in functioning both at work and in one's personal life. These estimated ten million Americans are said to be suffering from seasonal affective disorder, or SAD, a condition now widely accepted by the medical community and the public at large. Another 14 percent of the adult population in the United States is estimated to suffer from a lesser form of SAD, known as the winter blues. Sufferers of SAD have difficulty waking up in the morning and feel less cheerful, energetic, creative, and productive during the darker winter days than at other times of the year.

The description of SAD as a syndrome and the systematic development of light therapy to treat it took place at the National Institute of Mental Health (NIMH) in the early 1980s. Currently, the most commonly used method for administering light therapy is by means of a special light fixture or light box containing ordinary white fluorescent or full-spectrum fluorescent light bulbs that emit intensity of therapeutic ranges between 2,500 lux and 10,000 lux (lux being a measurement of intensity). The drawbacks of these light boxes include UV rays emanated from fluorescent light bulbs and the heat generated by fluorescent light bulbs. Another drawback, possible the most marked disadvantage, is revealing the fact that a person is administering light therapy to oneself-upon seeing the bizarre light box sitting on a table, people may make judgments about a person's competency or mental health. To ensure a patient's privacy, the times and places where light therapy is administered is therefore restricted.

One commonly used light box comprises of an array of fluorescent bulbs with a diffusing screen. The patient is exposed to the light by positioning himself or herself in proximity to the light box for a period of time ranging from about thirty minutes to several hours, depending on the level of illumination, light source, distance from illumination source, individual response, and the like. Treatments are generally administered on a frequent, often daily, basis. Research has suggested that the level of illumination is a critical aspect of the treatment. Some studies have suggested that the optimal level of illumination is one that matches the level of “natural” light shortly after sunrise or before sunset.

Conventional light boxes employing fluorescent tube illumination sources have several disadvantages. To achieve a sufficiently bright level of illumination, numerous fluorescent bulbs are required, and the light boxes consequently consume a significant amount of space and have a large footprint. They are also unattractive and unwieldy. Additionally, fluorescent light sources tend to flicker, causing variations in illumination output that can produce irritability, headache, and eye strain and, in severe cases, may cause migraine headaches. Fluorescent tubes also produce glare, which can induce unpleasant side effects such as headaches and eye strain.

Illumination devices that simulate sunrise or sunset, or some other natural lighting condition, are also used to treat patients having sleep disorders such as advanced or delayed sleep phase syndrome. Similarly, controlled exposure to bright light has been demonstrated to be effective for treating other types of problems such as jet lag, circadian rhythm abnormalities, and the like. Light sources used for treating sleep disorders are generally similar to the light boxes described above and suffer from similar disadvantages.

Illumination devices are also used in the practice of color therapy, which involves exposing patients to illumination of a selected wavelength band. Preferential exposure to selected wavelength light is believed to stimulate both metabolic and psychological responses. Illumination devices for use in color therapy generally employ incandescent, fluorescent or halogen light sources with filters to select for the appropriate wavelength band.

Another common way of administering light therapy is by utilizing a device for simulating summer dawn on a winter morning. Researchers have found that simulating the light of a summer dawn helps patients with SAD waking up in the morning and feeling better during the day. One such device is known as a dawn simulator, which is a small electronic gadget that can be plugged into an ordinary bedside lamp, allowing the light to be turned on in a graded way that is akin to the change in lighting occurring naturally during a summer dawn. Another such device is known as a sunrise clock, which combines a light source with an alarm clock and a mechanism geared to producing an artificial dawn. The disadvantage of both devices is that both devices work only with incandescent lamps, of which the intensity does not fall into the therapeutic range between 2,500 lux and 10,000 lux. Incandescent lamps are not recommended for light therapy.

Regarding other types of light therapy, it has been shown that color light may provide benefits to people. For example, blue light may be used to treat circadian disorders including SAD. There have been recent developments in LED technology known as RGB (red, green, blue) LED or full-color LED. These new LEDs can emit multiple colors and bright white light as well. Examples of such LEDs are available from Kingbright Corporation (225 Brea Canyon Road, City of Industry, Calif.).

### SUMMARY OF THE INVENTION

In many embodiments, the invention transforms a therapeutic light therapy device into an elegant looking table/floor lamp by means of LED technology. The device of the invention has a number of advantages over other light therapy devices on the market. For example, the device integrates a therapeutic light therapy function into a lamp. Patients can place and use the device anywhere, either at work or at home. By simply switching the lamp head to face the eyes, a patient can receive light therapy anytime. As the light-therapy function is concealed in a normal-looking lamp, speculation from others is avoided, and the privacy of a patient is maintained. In addition, in many embodiments the device uses LED lamps which do not emit UV rays and emit very little heat; therefore, the device can be safely used at almost any distance between the patient and the lamp.

According to other aspects of the invention, the device fills the gap between a dawn simulator and an optimal and effective light therapy device. The invention has a number of advantages over conventional dawn simulators on the market. For example, the device of the invention substantially simulates the change in lighting that occurs naturally during a summer dawn because LED lamps emits light that is more natural, bright, and white than that emitted by incandescent lamps. The eyes are typically more sensitive to bright light in the early morning hours; therefore, the device with bright LED is more effective than conventional devices.

In addition, because the device of the invention has a light-therapy function, by programming the device, a patient can receive light therapy upon waking and while still lying in bed. Studies have shows that receiving light therapy in the early morning is typically the most effective for a patient. Further, the device of the invention utilizes a unique hinge that enables the lamp to be fully adjustable. As such, the device is able to direct light onto the face efficiently for increased effectiveness.

Accordingly, although there are many light therapy light boxes and dawn simulators and sunrise alarm clocks on the market, the invention overcomes their respective drawbacks by integrating a light-therapy function and a dawn/dusk simulator with a reading lamp. The invention optimizes the function of light therapy and begins a new era of using light therapy to treat SAD and winter blues, disturbances of sleep cycles, such as jet lag, work shift fatigue, delayed sleep phase syndrome (DSPS), and advanced sleep phase syndrome (ASPS).

According to one aspect of the invention, a lamp for providing light therapy includes an input circuit, an output circuit, and a signal processing circuit. The input circuit may include a user input for providing control signals to the signal processing circuit based on input at the user input from a user. The output circuit may include a light output configured to emit light at a level of at least about 2,500 lux to 10,000 lux or more. The light output may include one or more white LEDs and may also include one or more color LEDs. The signal processing circuit is connected to the input circuit and the output circuit, and receives the control signals from the input circuit to control the output circuit based on the control signals.

According to another aspect of the invention, a memory may be provided in communication with the signal processing circuit. The memory may store a plurality of output modes, such as a light therapy mode. In embodiments in which the light output includes one or a few high-intensity LEDs, the light output may include a diffuser that is configured to diffuse the high-intensity light for effective use.

Other features and advantages of the present invention will become apparent to those skilled in the art from a consideration of the following detailed description taken in conjunction with the accompanying drawings.

### BRIEF DESCRIPTION OF THE SEVERAL VIEWS OF THE DRAWINGS

FIGS. 1A, 1B, and 1C are front, side, and top views of a programmable multifunction lamp according to a number of embodiments;

FIG. 2 illustrates a control panel and a display of the lamp;

FIG. 3 illustrates an LED array of the lamp;

FIG. 4 is a block diagram of electrical components of the lamp;

FIG. 5 is a schematic diagram of a control unit and associated circuitry;

FIG. 6 is a schematic diagram of an AC/DC power adapter;

FIG. 7 is a schematic diagram of DC power supply;

FIG. 8 is a schematic diagram of an LED array;

FIG. 9 illustrates programming methodology for the multifunction lamp;

FIG. 10 is a block diagram of a lamp for providing light therapy according to a number of embodiments;

FIG. 11 illustrates an example of a diffuser; and

FIG. 12 illustrates an example of a diffusion pattern of a diffuser.

### DETAILED DESCRIPTION OF THE INVENTION

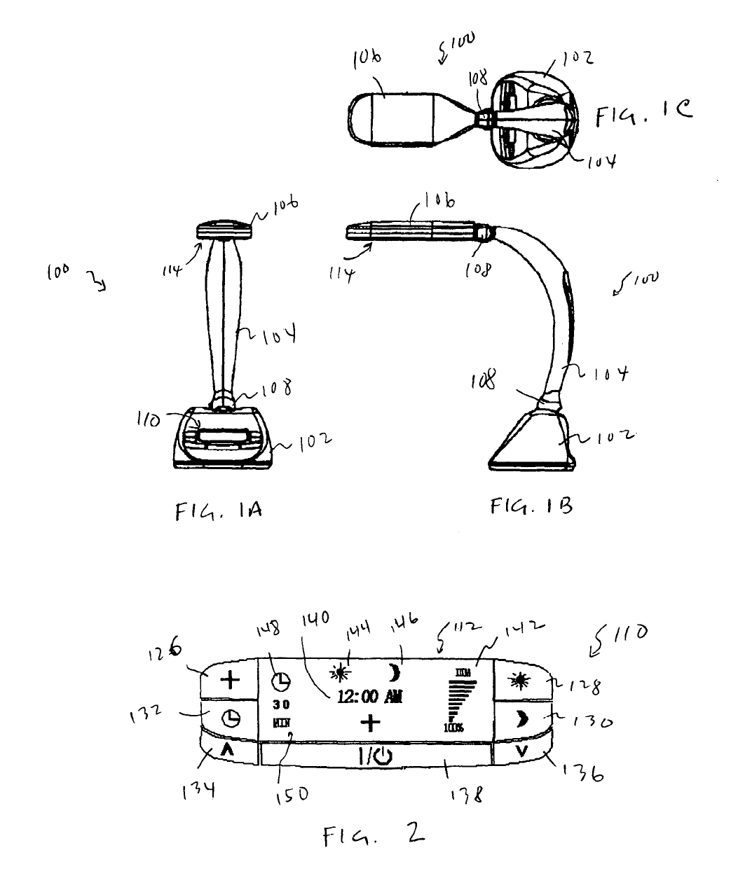

Referring to the drawings, a multifunction lamp 100 is illustrated in FIGS. 1A, 1B, and 1C. According to a number of embodiments, the lamp 100 may be easily programmed for automatic sunrise and sunset. Over a specified period of time (for example, 30 minutes), the lamp 100 gradually increases in brightness from barely visible to 100%. The lamp 100 may be set to start one-half hour before a person awakes to simulate a sunrise. After the specified period of time, the lamp 100 gradually brightens to full intensity, and an alarm may sound to awaken a user. At bedtime, a sunset simulation mode may be initiated by pressing a sunset button. The lamp 100 may then gradually dim until completely turning off at a specified time.

The multifunction lamp 100 may be beneficial to those who struggle to get up in the morning. The sunrise/sunset simulator functions promote a healthy sleep cycle, allowing a user to arise refreshed and energetic. When jolted out of bed by annoying alarm clocks, a person's sleep cycle is interrupted, leaving the person feeling tired. Clinical studies have shown dawn rise devices aid in the sleep/waking process, particularly for those people who work unusual hours. The dawn simulator alarm clock wakens a person gradually, allowing the person's sleep pattern to completely cycle.

According to other embodiments, the multifunction lamp 100 may be beneficial for those who are light deprived and do not receive sufficient natural sunlight in their lives. By programming the lamp 100, a user can have bright-light treatment (e.g., 10,000 lux) upon waking up and while lying in bed. Studies have shown that bright light in the morning is the most effective way to treat seasonal affective disorder (SAD) and winter blues. By pressing a timer button on the lamp 100, the duration of the treatment may be set. By pressing a dim button, the treatment intensity may be set.

According to still other embodiments, the lamp 100 includes a bright white LED array that is able to produce an incredibly bright, focused and evenly distributed beam of illumination without falloff or “soft edges” associated with incandescent lights. Reading and working under bright white illumination is easier on the eyes, while objects illuminated by bright white LEDs appear sharper with greater contrast. In addition, light from an LED does not have the yellow color associated with incandescent lights or the blue color associated with fluorescent lights. Further, colors appear brighter and more like natural daylight streaming in from a window than any other type of light bulb.

Moreover, LEDs consume about 1/10th the power of conventional incandescent lights, while each individual bright white LED is up to 10 times brighter than other bulbs. LEDs can also last a lifetime without the need for replacement. In many embodiments, the lamp 100 may include a panel of about 100 bright white LEDs. As bright white LEDs produce almost no heat, the head of the lamp 100 remains cool to the touch.

Turning to the description of the lamp 100 in more detail, Referring to the drawings, a multifunction lamp 100 according to a number of embodiments is illustrated in FIGS. 1A, 1B, and 1C. In many embodiments, the lamp 100 may include a base 102, an arm 104, and a light head 106 pivotally connected together with hinges 108. As shown in FIG. 2, a control panel 110 with a plurality of buttons and a display 112 may be provided on the base 102.

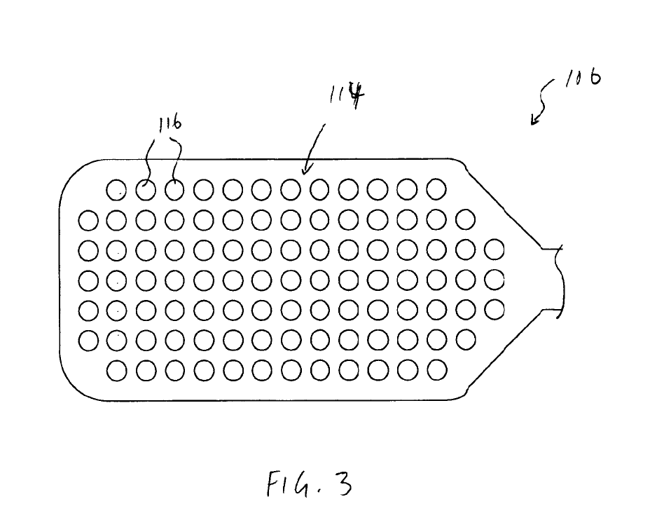

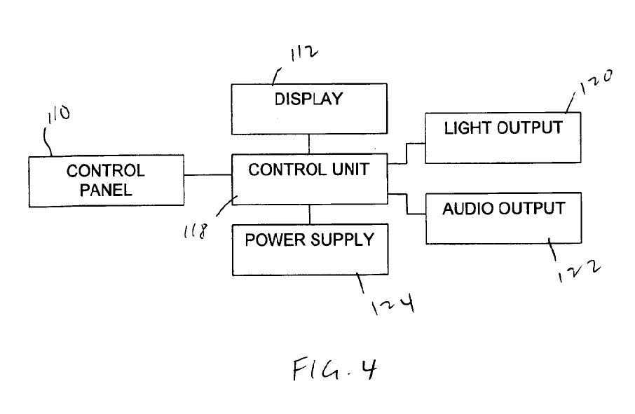

As shown in FIG. 3, the light head 106 may include an array 114 of light-emitting diodes (LEDs) 116. In many embodiments, the array 114 may include at least 75 LEDs 116. In other embodiments, the array 114 may include about 90 or more LEDs 116. In other embodiments, the LED array 114 includes a suitable number of LEDs 116 with a particular individual intensity so that the array 114 may emit at least about 10,000 lux. For example, in some of the embodiments, the array 114 may include about 30 or more LEDs 116, and in still other embodiments, the array 114 may include only a few LEDs 116, particularly for low-intensity light-therapy function. In still other embodiments, the array 114 may include a single high-intensity LED 116.

The hinge or socket 108 between the base 102 and the arm 104 allows the arm 104 to be moved or rotated with respect to the base 102. In addition, the hinge or socket 108 between the arm 104 and the head 106 allows the head 106 to be moved or rotated with respect to the arm 104. Accordingly, the head 106 and, thus, the LED array 114 may be moved about three axes and positioned at any desired location with respect to the base 102.

As shown in FIG. 4, the lamp 100 may include a control unit 118 connected to the control panel 110 and the display 112. The control unit 118 may include circuitry and be configured for performing a plurality of light functions. In this regard, a light output 120 may include the LED array 114, in addition to other light outputs not shown in the drawings.

In other embodiments, the control unit 118 may also include circuitry and be configured for performing a plurality of audio functions. In this regard, the lamp 100 may include an audio output 122 that may include one or more speakers (which is shown on a schematic diagram to follow). In addition, a power supply 124 may be provided for converting 120 VAC power to a DC power suitable for driving the LED array 114 and related circuitry.

As shown in FIG. 2, in many embodiments the control panel 110 may include a number of keys or switches, e.g., a light-therapy button 126, a dawn-simulator button 128, a dusk-simulator button 130, a timer 132, an increase button 134, a decrease button 136, and an ON/OFF button 138 that may also serve a snooze function. Also shown in FIG. 2, in many embodiments the display 112 may display a number of different information items and modes, e.g., a clock 140, an intensity meter 142, a dawn indicator 144, a dusk indicator 146, a timer 148, and a count-down indicator 150.

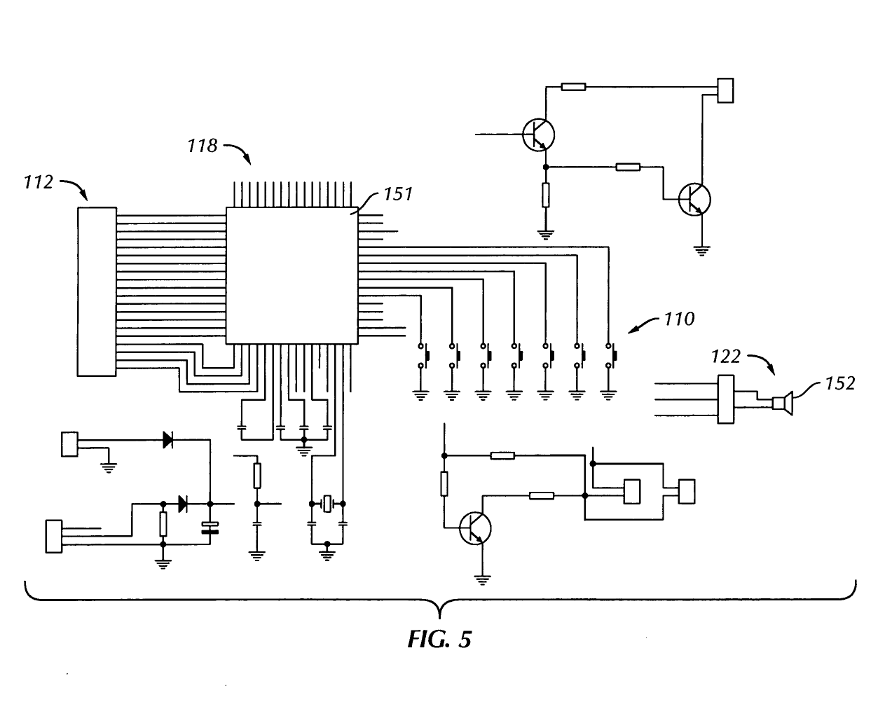

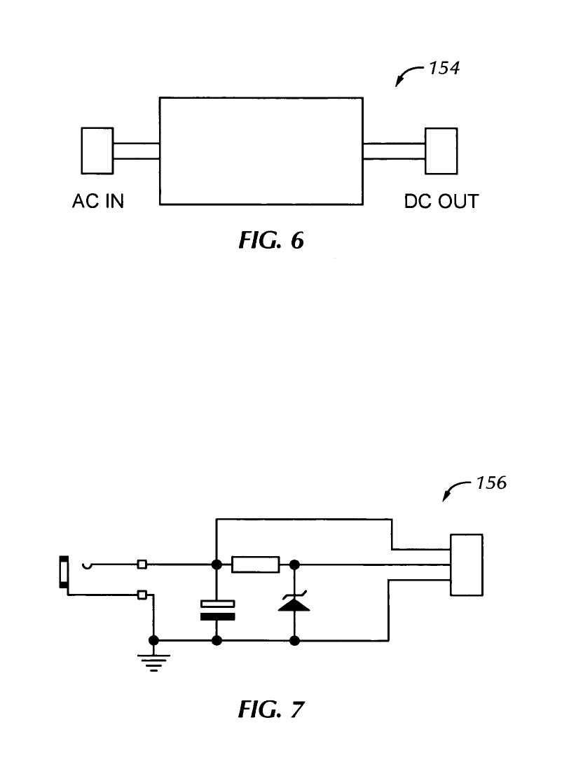

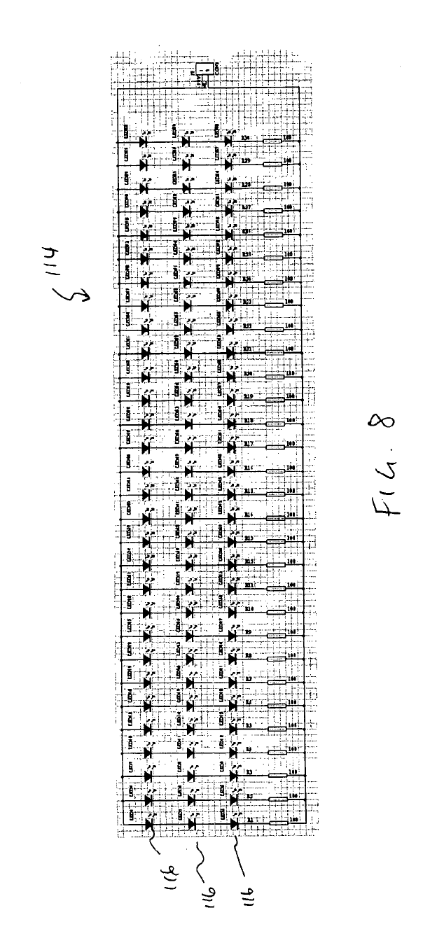

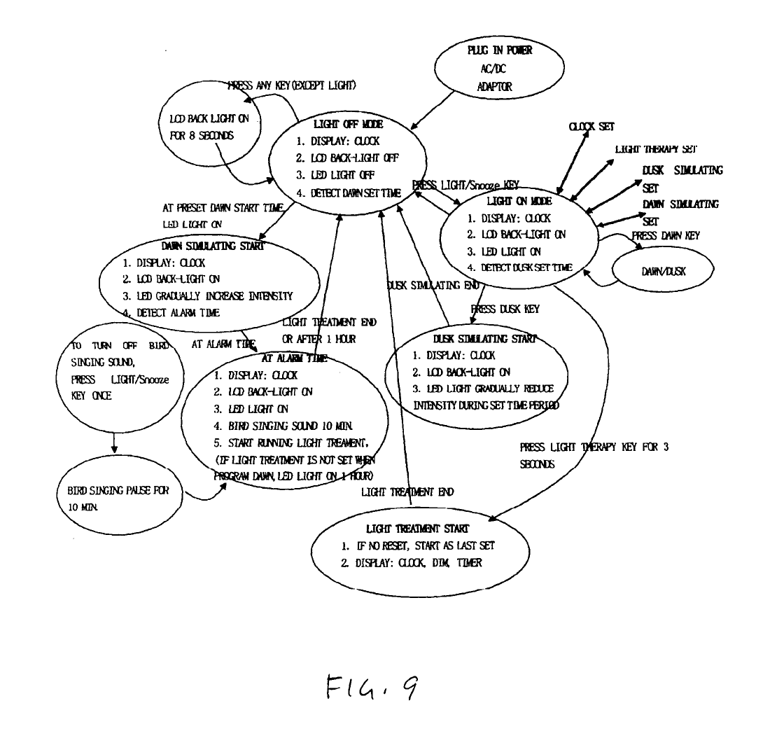

According to a commercial embodiment of the lamp 100, a schematic diagram of the control unit 118 is illustrated in FIG. 5, which may include a programmable integrated circuit (IC) 151. As shown, the control panel 110 with a plurality of switches may enable a user to provide input to the control unit 110. In addition, the display 112 may include a liquid-crystal display (LCD) as shown. Also shown is the audio output 122 including a speaker 152. FIGS. 6 and 7 illustrate schematic examples of an AC-to-DC power converter 154 and a DC power supply 156, respectively. FIG. 8 illustrates one of the embodiments of the LED array 114.

Reference is made to FIG. 9 in describing the operation of the lamp 100. After connecting the lamp 100 to a power supply, e.g., by plugging an AC/DC adaptor 154 (or alternatively, universal power adaptor) into a power outlet or by placing batteries into a battery compartment, the ON/OFF switch 138 may be actuated. To set the clock 140, the timer button 132 may be pressed and held until an hour indicator (e.g., on the clock 140) blinks, with the increase or decrease buttons 134 or 136 then being actuated to set the hour. The same may be done for a minute indicator (e.g., on the clock 140), with the time then saved.

To set a light-therapy program, the light-therapy button 126 may be pressed until the timer indicator 148 blinks. The increase and decrease buttons 134 and 136 may then be pushed to set the timer to the desired duration. The intensity meter 142 may then blinks, with the increase and decrease buttons 134 and 136 pushed to set the desired light intensity. The settings may then be saved.

To set a dawn-simulation function, the dawn-simulation button 128 may be pressed until an hour indicator blinks, with the increase and decrease buttons 134 and 136 being pushed to set a desired alarm hour, with the same then being done for the desired minutes. The timer indicator 148 may then blink, with the increase and decrease buttons 134 and 136 being pushed to set an advanced dawn beginning time (i.e., the time prior to the set alarm time). The user may then be prompted to choose whether or not a preset light-therapy program should initiate after the alarm has gone off.

To set a dusk-simulation function, the dusk-simulation button 130 may be pressed until the timer indicator 148 blinks, with the increase and decrease buttons 134 and 136 then pushed to set a dusk duration time, with the program then saved.

To initiate a preset light-therapy program, the user may position the head 106 so that the LED array 114 is directed to the user's face. The light-therapy button 126 may then be pressed to begin the light therapy, with the timer 150 beginning to count down, with the therapy session running at the preset parameters (i.e., duration and intensity).

To initiate a dawn-simulation program, the dawn-simulation button 128 may be pressed. Thereafter, the LED array 114 will turn on automatically at the preset advanced time before the preset alarm time. The light intensity will gradually increase to 100% of the preset level when reaching the alarm time. When reached, an alarm sound will sound (e.g., a beeper or a simulated bird singing or any natural soothing sound). If a light-therapy program was preset in the dawn-simulation program, then a light-therapy program may begin automatically. If not present, the lamp 100 can turn off in predetermined time. Alternatively, the ON/OFF button 138 (or snooze button) may be pressed once to turn off the alarm sound and twice to turn off the LED array 114.

To initiate a dusk-simulation program, the dusk-simulation button 130 may be pressed. The intensity of the LED array 114 will gradually decrease according to the preset parameters until the LED array 114 turns off automatically. To utilize the lamp 100 as a light, the ON/OFF button 138 may be pressed anytime.

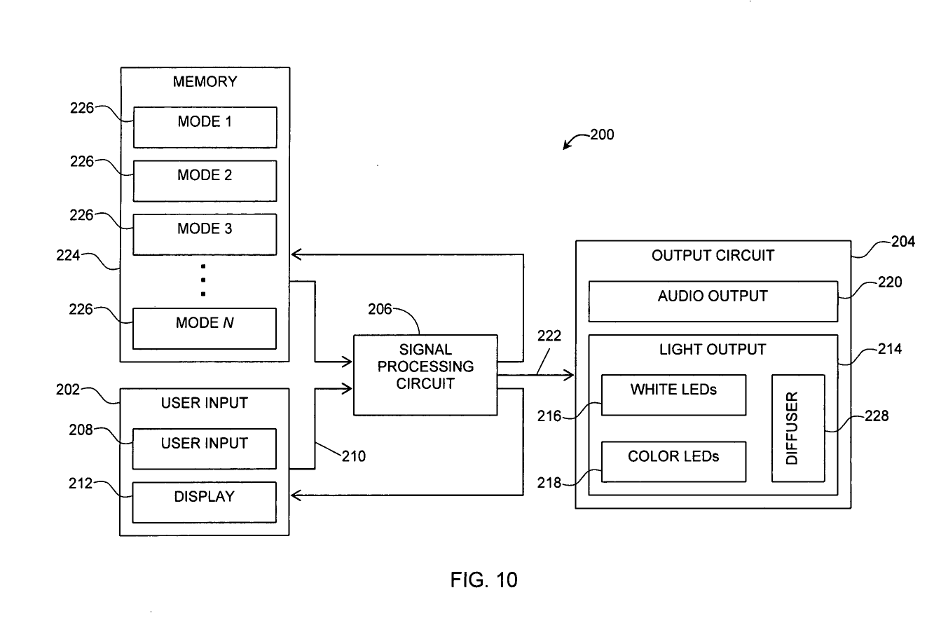

According to a number of embodiments as exemplified in FIG. 10, a lamp 200 for providing light therapy may include an input circuit 202, an output circuit 204, and a signal processing circuit 206. The input circuit 202 may include a user input 208 such as a keypad as described above that is configured to enable a user to provide control signals 210 to the signal processing circuit 206. The input circuit 202 may also include a display 212 as described above.

In some of the embodiments, the output circuit 204 may include a light output 214 that is configured to emit light at a predetermined level, such as at least about 2,500 lux. In other embodiments, the processing circuit 206 may cause the light output 214 to emit light up to levels of 10,000 lux or more. The light output 214 may include one or more LEDs as described above, including white LEDs 216 and color LEDs 218 (e.g., red, green, and blue) depending upon the embodiment. In other embodiments, the output circuit 220 may also include an audio output 220 such as a speaker.

The signal processing circuit 206 is in communication with or connected to the input circuit 202 and the output circuit 204, and provides output signals 222 to the output circuit 204 based either on the control signals 210 from the input circuit 202 or on one or more output modes, which is discussed below.

In a number of embodiments, the lamp 200 may include a memory 224 in communication with the signal processing circuit 206. The memory 224 may store a plurality of output modes 226, e.g., MODE 1, MODE 2, MODE 3, . . . , MODE N. The output modes 226 may be set and stored by a user at the user input 208, or one or more of the output modes 226 may be already stored in the memory 224 prior to commercial sale.

The signal processing circuit 206 may provide the output signals 222 to the output circuit 204 based on one of the modes 226. For example, the plurality of modes 226 may include a light therapy mode. According to a light therapy mode, the signal processing circuit 206 may cause the light output 214 to emit light at a predetermined effective level of at least about 2,500 lux for a predetermined amount of time, such as about 30 minutes. Depending upon a particular user's need, the processing circuit 206 may cause the light output 214 to emit light at other predetermined effective levels, either higher or lower, and for other predetermined amounts of time. For example, a light therapy mode 226 may cause the light output 214 to emit light from the white LEDs 216 at an effective level of at least 5,000 lux for about 15 minutes. Those skilled in the art will appreciate that other combinations or gradations of emitted light levels and periods of time may be implemented.

Generally speaking, the signal processing circuit 206 and the light output 214 may be configured to emit light at a level capable of providing light therapy. Accordingly, for the purposes of this description, the term “a level capable of providing light therapy” shall be defined as a level of light that is able to effectively provide light therapy to a person for a given period of time. For example, exposing a person to 2,500 lux for about 30 minutes provides effective light therapy.

As another example, the plurality of modes 226 may include a dawn simulator mode which causes the signal processing circuit 206 to control the output circuit 204 as a dawn simulator. In addition, the modes 226 may include color output modes that are intended to create desired moods, or color therapy. For example, one of the modes 226 may cause the color LEDs 218 to emit green light, which may boost energy of a user. Alternatively, one of the modes 226 may cause the color LEDs 218 to emit red light, which may create a passionate setting. An orange output mode 226 may cause an increase in appetite, and a blue output mode 226 may create a calm and relaxing mood.

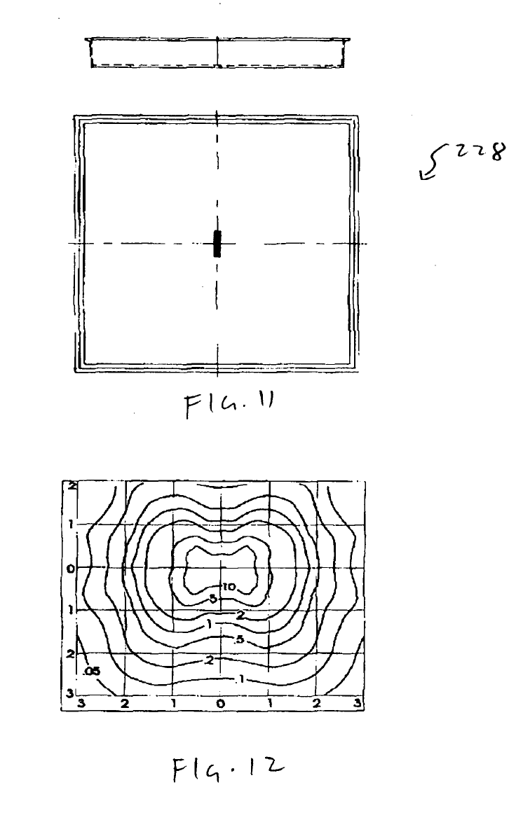

In some of the embodiments, the light output 214 may include a single high-intensity white LED 216 that is capable of providing light at an effective light therapy level of about 2,500 lux. Because of the intensity of a single LED, the output circuit 204 may include a diffuser 228, an example of which is shown in FIG. 11. The diffuser 228 is configured to diffuse the high-intensity light emitted from a single LED 216 as shown by the example of the diffusion pattern of FIG. 12. Accordingly, light from just one or a few high-intensity LEDs 216 passing through the diffuser 228 may be effectively used according to a desired purpose.

Those skilled in the art will understand that the preceding embodiments of the present invention provide the foundation for numerous alternatives and modifications thereto. For example, in addition to utilizing batteries or an AC adapter, the lamp 100 may be plugged directly into a 120-volt, 60-cycle power outlet for a power supply. These other modifications are also within the scope of the present invention. Accordingly, the present invention is not limited to that precisely as shown and described in the present invention.

What is claimed is:

1. A lamp capable of providing light therapy, the lamp comprising:
an input circuit including a user input and for providing control signals based on input at the user input from a user;
an output circuit including a light output configured to emit light at a level capable of providing light therapy; and
a signal processing circuit connected to the input circuit and the output circuit, and for receiving the control signals from the input circuit and for controlling the output circuit based on the control signals.

2. The lamp of claim 1 wherein the light output is configured to emit light at a level of at least about 2,500 lux.

3. The lamp of claim 1 wherein the light output is configured to emit light at a level of at least about 10,000 lux.

4. The lamp of claim 1 further comprising a memory in communication with the signal processing circuit and for storing a plurality of output modes.

5. The lamp of claim 4 wherein the plurality of output modes includes a light therapy mode.

6. The lamp of claim 5 wherein the light therapy mode causes the signal processing circuit to cause the light output emit light at a level of at least about 2,500 lux for a predetermined period of time.

7. The lamp of claim 6 wherein the light therapy mode causes the signal processing circuit to cause the light output emit light at 2,500 lux for about 15 minutes.

8. The lamp of claim 1 wherein the light output includes at least one light-emitting diode (LED).

9. The lamp of claim 8 wherein the light output includes a diffuser.

10. The lamp of claim 8 wherein the light output includes a plurality of LEDS wherein the plurality of LEDs includes a number of LEDs capable of emitting color.

11. The lamp of claim 1 wherein the output circuit includes an audio output.

12. The lamp of claim 1 wherein the input circuit includes a display.

13. A method for providing light therapy, the method comprising:
providing a lamp including:
an input circuit including a user input and for providing control signals based on input at the user input from a user;
an output circuit including a light output configured to emit light at a level capable of providing light therapy; and
a signal processing circuit connected to the input circuit and the output circuit, and for receiving the control signals from the input circuit and for controlling the output circuit based on the control signals; and
causing the light output to emit light at a predetermined effective level for a predetermined amount of time.

14. The method of claim 13 wherein the causing step further comprises causing the light output to emit light at a predetermined effective level of at least about 2,500 lux.

15. The method of claim 14 wherein the causing step further comprises causing the light output to emit light at the predetermined effective level for a predetermined amount of time of at least about 15 minutes minutes.

16. A lamp for providing light therapy, the lamp comprising:
an input circuit including a user input and for providing control signals based on input at the user input from a user;
an output circuit including a light output configured to emit light at a level of at least 2,500 lux, the light output including at least one white LED;
a signal processing circuit connected to the input circuit and the output circuit, and for receiving the control signals from the input circuit and for controlling the output circuit based on the control signals; and
a memory in communication with the signal processing circuit and for storing a plurality of output modes including a light therapy mode.

17. The lamp of claim 16 wherein the light output includes a diffuser.

18. The lamp of claim 16 wherein the light output includes a plurality of LEDS capable of emitting color.

19. The lamp of claim 16 wherein the output circuit includes an audio output.

20. The lamp of claim 16 wherein the input circuit includes a display.
